(19) United States
(12) Patent Application Publication
(10) Pub. No.: US 2004/0132099 A1
(43) Pub. Date: Jul. 8, 2004

(54) Methods, systems and computer software for designing and synthesizing sequence arrays

(75) Inventor: Earl A. Hubbell, Mountain View, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/627,271

(22) Filed: Jul. 25, 2003

Related U.S. Application Data
(62) Division of application No. 09/640,962, filed on Aug. 16, 2000, now abandoned.
(60) Provisional application No. 60/149,510, filed on Aug. 17, 1999.
(60) Provisional application No. 60/182,288, filed on Feb. 14, 2000.

(51) Int. Cl. G01N 33/53; G06F 19/00; G01N 33/48; G01N 33/50; G01N 31/00; G01N 33/543

(52) U.S. Cl. 435/7.1; 436/518; 702/19; 702/22

(57) ABSTRACT

Embodiments of the invention provides methods, computer software products and systems for arranging polymers during combinatorial polymer synthesis so that the border or edge between synthesis site is minimized. In one embodiment, travelling salesman algorithm is used to minimize the edges. In another embodiment, a locally greedy optimization method is provided. In addition, methods and software products are provided for solving the robust arrangement problem for multi-probe gene expression arrays.

### CROSS-REFERENCE TO RELATED APPLICATIONS

This application claims the priority of U.S. Provisional Applications, Serial No. 60/149,510, filed on Aug. 17, 1999, titled “Edge Minimization” and Serial No. 60/182,288, filed on Feb. 14, 2000, titled “Lithographic Mask Design and Synthesis of Diverse Probes on a Substrate.” The 60/149,510 and 60/182,288 applications are incorporated in their entity herein by reference for all purposes.

### COPYRIGHT NOTICE

A portion of the disclosure of this patent document contains material that is subject to copyright protection. The copyright owner has no objection to the xerographic reproduction by anyone of the patent document or the patent disclosure in exactly the form it appears in the Patent and Trademark Office patent file or records, but otherwise reserves all copyright rights whatsoever.

### APPENDIX

Appendices A and B are included herewith and form a part of the disclosure.

### BACKGROUND OF THE INVENTION

U.S. Pat. No. 5,424,186 describes a pioneering technique for, among other things, forming and using high density arrays of molecules such as oligonucleotide, RNA, peptides, polysaccharides, and other materials. This patent is hereby incorporated by reference for all purposes. Arrays of oligonucleotides or peptides, for example, are formed on the surface by sequentially removing a photoremovable group from a surface, coupling a monomer to the exposed region of the surface, and repeating the process. These techniques have been used to form extremely dense arrays of oligonucleotides, peptides, and other materials. Such arrays are useful in, for example, drug development, gene expression monitoring, genotyping, and a variety of other applications. The synthesis technology associated with this invention has come to be known as “VLSIPS™” or “Very Large Scale Immobilized Polymer Synthesis” technology. Despite the great success of the technique disclosed in the U.S. Pat. No. 5,434,186, there is still a need for improved methods for large scale synthesis of polymers.

### SUMMARY OF THE INVENTION

According to some aspects of the invention, methods, systems, and computer software are provided for improving the arrangement of specified features within complex patterns. One aspect of the invention concerns arranging the specified features to have a reduced number of differences between adjacent features (edges). The methods, systems, and computer software products are particularly suitable for designing and forming sequence arrays such as nucleic acid or peptide arrays.

In one aspect of the invention, computer implemented methods for arranging polymers for combinatorial synthesis of said polymers on a substrate are provided. In some embodiments, computer-implemented optimization steps for performing a travelling salesman optimization are performed to arrange polymers in an order such that when such polymers are assigned spatial locations for synthesis, edge counts between synthesis sites are reduced to reduce errors during photodirected synthesis, such as diffraction, internal reflection, and scattering. As used herein, the term edge-count may be a weighted edge-count taking into account distances to cells leaking radiation.

In one particularly preferred embodiment of the invention, this travelling salesman optimization is carried out using a locally greedy insertion algorithm, although many other methods for performing a travelling salesman optimization are also suitable for at least some embodiments of the invention.

In another aspect of the invention, computer implemented methods for transforming a pre-existing assignment of polymers to spatial locations for synthesis into an assignment of polymers to spatial locations with reduced edge counts. In a preferred embodiment, such methods use a locally greedy algorithm to choose new spatial locations for the polymers. In a preferred embodiment, a locally greedy optimization is performed on either polymers or blocks of polymers. In some embodiments, the locally greedy optimization involves dividing polymers into a plurality of blocks, wherein, each of the blocks contains one or more related polymers, and each of the blocks is to be assigned to one corresponding slot on the substrate, where a slot is a plurality of locations sufficient to contain the polymers in a block. The process may be repeated until all blocks are assigned. In a preferred embodiment, the blocks are first ordered randomly, to avoid poor initial arrangements of polymers. In the preferred embodiment, a subset of the blocks from the set of currently unassigned blocks is selected, usually starting from the first unassigned block. The number of blocks in the subset may be adjusted by the user. Preferred ranges may include, 5-20, 20-100,100-500, 500-1000, 1000-10000, 10000-100000 blocks in a subset. Such ranges may be chosen by the user to adjust, for example, the running time of the methods. One block of the subset is assigned to an empty slot if this block is the block whose assignment to the empty slot results in the least edge count of all blocks possibly assigned to the slot.

This method is particularly useful for arranging oligonucleotide probes in a nucleic acid array that is manufactured using photodirected combinatorial synthesis using a set of masks or computer controlled micromirrors.

In another aspect of the invention, computer software products for arranging polymers for combinatorial synthesis of polymers on a substrate are provided. The computer software product contains: 1) computer program code for performing a travelling salesman optimization to arrange polymers in an order such that when such polymers are assigned spatial locations for synthesis, edge counts between synthesis sites are reduced; and 2) a computer readable medium for storing the codes.

In another aspect of the invention, computer software products for transforming a pre-existing assignment of polymers to spatial locations for synthesis into an assignment of polymers to spatial locations with reduced edge counts are provided. The computer software product contains computer program code for performing a locally greedy algorithm for assigning polymers to spatial locations, and a computer readable medium for storing the codes. In a preferred embodiment, the computer software product contains program code for performing locally greedy optimization including computer program code for dividing polymers into a plurality of blocks, computer program code for unassigning such blocks from their current spatial locations, computer program code for selecting a subset of the blocks from unassigned blocks, and computer program code for assigning one block of the set to an empty slot if the block results in a least edge count among the blocks of the subset.

The computer software product may also contain program code for repeating the steps of selecting and assigning until all blocks are assigned. In some preferred embodiments, the computer software product may contain computer program code for randomly ordering unassigned blocks, and may contain computer software code for accepting a number of blocks in a subset.

Furthermore, a computer implemented method for robust arrangement problem (RAP) is also provided. Oligonucleotide arrays for monitoring gene expression may have certain number of probe pairs or probes devoted to any given gene. Local problems (flecks of dust, bubbles, defects) may occur on the array, and if the probes (pairs) are arranged adjacent to each other (these probes may be referred hereafter as non-robust, bad or adjacent), there may be no informative probes remaining for that gene if a defect occurs. The RAP is a probe distribution problem of arranging all the probes (pairs) on the chip, so that of the N (typically, 10, 15 or 20 pairs) probes (pairs) associated with any given gene, no more than K, such as 2, 3, 4 or 5, of them are within a radius R of each other.

In some embodiments, all non-robust probe pairs are removed from the chip as blocks, leaving empty slots behind, and an equal number of robust probe pairs are chosen randomly and also removed, and then these blocks are replaced (almost) randomly into the slots, the number of new non-robust blocks will be reduced greatly (typically again cut to 1% of the former value). Computer software products containing code for performing the RAP steps are also provided. In preferred embodiments, a polymer (probe) arrangement software product performs the edge minimization and solves RAP.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 illustrates an example of a computer system that may be utilized to execute the software of an embodiment of the invention.

FIG. 2 illustrates a system block diagram of the computer system of FIG. 1.

FIG. 3 shows a process for a locally greedy optimization.

FIG. 4 shows a process for using one embodiment of the software product of the invention.

### DESCRIPTION OF THE PREFERRED EMBODIMENTS

Reference will now be made in detail to the preferred embodiments of the invention. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention.

As will be appreciated by one of skill in the art, the present invention may be embodied as a method, data processing system or program products. Accordingly, the present invention may take the form of data analysis systems, methods, analysis software and etc. Software written according to the present invention is to be stored in some form of computer readable medium, such as memory, hard-drive, DVD ROM or CD ROM, or transmitted over a network, and executed by a processor.

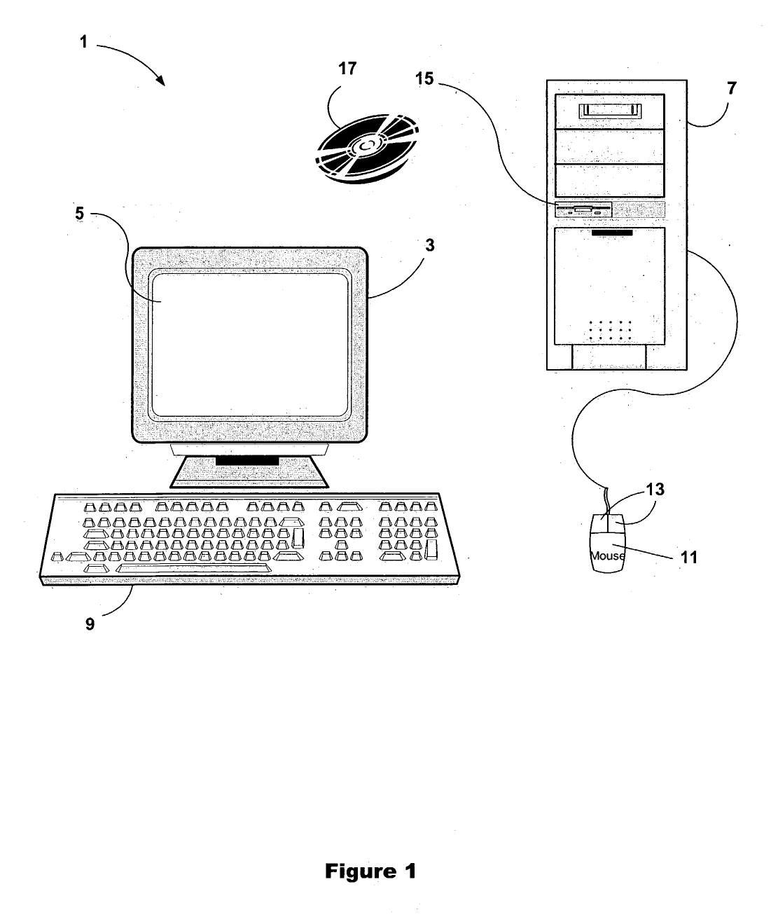

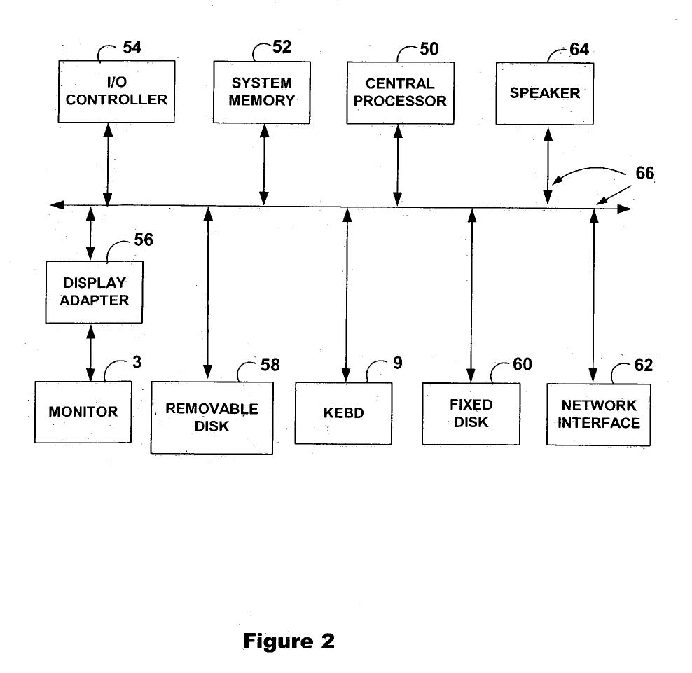

FIG. 1 illustrates-an example of a computer system that may be used to execute the software of an embodiment of the invention. FIG. 1 shows a computer system 1 that includes a display 3, screen 5, cabinet 7, keyboard 9, and mouse 11. Mouse 11 may have one or more buttons for interacting with a graphic user interface. Cabinet 7 preferably houses a CD-ROM or DVD-ROM drive 13, system memory and a hard drive (see, FIG. 2) which may be utilized to store and retrieve software programs incorporating computer code that implements the invention, data for use with the invention and the like. Although a CD 15 is shown as an exemplary computer readable medium, other computer readable storage media including floppy disk, tape, flash memory, system memory, and hard drive may be utilized. Additionally, a data signal embodied in a carrier wave (e.g., in a network including the internet) may be the computer readable storage medium.

FIG. 2 shows a system block diagram of computer system 1 used to execute the software of an embodiment of the invention. As in FIG. 1, computer system 1 includes monitor 3, and keyboard 9, and mouse 11. Computer system 1 further includes subsystems such as a central processor 51, system memory 53, fixed storage 55 (e.g., hard drive), removable storage 57 (e.g., CD-ROM), display adapter 59, sound card 61, speakers 63, and network interface 65. Other computer systems suitable for use with the invention may include additional or fewer subsystems. For example, another computer system may include more than one processor 51 or a cache memory. Computer systems suitable for use with the invention may also be embedded in a measurement instrument or performed using ASIC devices or the like.

In one aspect of the invention, methods, systems and computer software products are provided to minimize the edges between features in a photo-lithograhic synthesis of polymers.

Methods of forming high density arrays of oligonucleotides, peptides and other polymer sequences with a minimal number of synthetic steps are disclosed in, for example, U.S. Pat. Nos. 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668 and U.S. Pat. No. 5,677,195 which disclose methods of forming vast arrays of peptides, oligonucleotides and other molecules using, for example, light-directed synthesis techniques. See also, Fodor et al., Science, 251, 767-77 (1991). These procedures for synthesis of polymer arrays are now referred to as VLSIPS™ procedures. Using the VLSIPS™ approach, one heterogeneous array of polymers is converted, through simultaneous coupling at a number of reaction sites, into a different heterogeneous array. See, U.S. Pat. Nos. 5,384,261 and 5,677,195.

The development of VLSIPS™ technology as described in the above-noted U.S. Pat. No. 5,143,854 and PCT patent publication Nos. WO 90/15070 and 92/10092, is considered pioneering technology in the fields of combinatorial synthesis and screening of combinatorial libraries.

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In the event that an oligonucleotide analogue with a polyamide backbone is used in the VLSIPS™ procedure, it is generally inappropriate to use phosphoramidite chemistry to perform the synthetic steps, since the monomers do not attach to one another via a phosphate linkage. Instead, peptide synthetic methods are substituted. See, e.g., Pirrung et al. U.S. Pat. No. 5,143,854.

Peptide nucleic acids are commercially available from, e.g., Biosearch, Inc. (Bedford, Mass.) which comprise a polyamide backbone and the bases found in naturally occurring nucleosides. Peptide nucleic acids are capable of binding to nucleic acids with high specificity, and are considered “oligonucleotide analogues” for purposes of this disclosure.

In addition to the foregoing, additional methods which can be used to generate an array of oligonucleotides on a single substrate are described in PCT Publication No. WO 93/09668. In the methods disclosed in the application, reagents are delivered to the substrate by either (1) flowing within a channel defined on predefined regions or (2) “spotting” on predefined regions or (3) through the use of photoresist. However, other approaches, as well as combinations of spotting and flowing, may be employed. In each instance, certain activated regions of the substrate are mechanically separated from other regions when the monomer solutions are delivered to the various reaction sites.

As described above, one method of synthesizing an oligonucleotide array or peptide array is by a photolithographic VLSIPS™ method. In this method, light is used to direct the synthesis of oligonucleotides in an array. In each step, light is selectively allowed through a mask to expose cells in the array, activating the oligonucleotides in that cell for further analysis. For every synthesis step, there is a mask with corresponding open (allowing light) and closed (blocking light) cells. Each mask corresponds to a step of combinatorial synthesis. This method is useful for synthesizing many different types of polymers including oligonucleotides (often used as probes against nucleic acid target), peptides and polysaccharides. However, for the purpose of clarity, various aspects of the invention are described using exemplary embodiments for synthesizing oligonucleotide probes.

As used herein, edges are the differences between polymer synthesis sites. In some embodiments, edges are difference between the synthesis steps used for one probe and the synthesis steps used for another probe. Due to reflection, internal reflection, scattering and other effects during photodirected synthesis, light does not precisely fill the areas designed to be illuminated. Light often leaks from these areas into nearby regions. Every edge is a possibility for light leakage, which may lead to a lower quality set of probes being synthesized. It is desirable to minimize such unintended illumination.

Edge counts may be integers: zero, one, or any other number. Because light leakage may occur over long distances (60 microns), in some instances it may be desirable to obtain a weighted edge count (WEIGHTED EDGE COUNT) taking into account the distance to the cell leaking light. For example, if the light leakage halves every 10 microns, and features are 20 microns across, then it is reasonable to weight the edges between a target cell and a cell one feature distant as ¼ the edges of the cell immediately adjacent to the target cell.

One of skill in the art would appreciate that this is one of many possible weighting functions. Other weighing functions are also within the scope of the invention. For computational efficiency, in one embodiment, only nearby cells need to be counted, since weights for extremely distant cells are negligible.

In one aspect of the invention, methods and computer software products are provided to arrange the probes in an order such that the total edge count between probes adjacent in the order are reduced. In a synthesis scheme of N synthesis steps, each probe can be viewed as a binary vector of length N. The number of edges between two probes is the number of places where the binary vectors are different, the so called Hamming distance. If an ordered list of probes are assigned to spatial positions in such a manner that are typically probes adjacent in the list are adjacent on the chip, then the number of edges on the chip will be similar to the number of edges in the list. Thus, finding an ordering of the vectors in the list so that the total distance between all adjacent vectors is minimal will provide a reduced set of edges on the chip. In some embodiments of the invention, an ordering of the list is provided by performing travelling salesman optimization. In one embodiment, a locally greedy insertion heuristic is used to construct the ordered list.

As used herein, the term travelling salesman optimization refers to methods, steps, algorithm, solution or the like for performing optimization (particularly minimization) that are also useful for solving the travelling salesman problem. Many well known approximate solutions, methods, steps and algorithms have been developed to perform travelling salesman problem in the art (see, e.g., David Applegate, Robert Bixby, Vasek Chvátal, and William Cook, On the solution of travelling salesman problems, Documenta Mathematica, vol. 3, pp. 645-656, 1998. Extra volume ICM 1998; David Applegate, Robert Bixby, Vasek Chvátal, and William Cook, Finding tours in the tsp, Tech. Rep. TR99-05, Department of Computational and Applied Mathematics, Rice University, 1999; Leonard M. Adleman, Molecular computation of solutions to combinatorial problems, Science, vol. 266, pp. 1021-1024, 1994; Norbert Ascheuer, Matteo Fischetti, and Martin Grötschel, A polyhedral study of the asymmetric travelling salesman problem with time windows. Available via WWW at tt www.zib.de, Feb. 1997. Preprint; Norbert Ascheuer, Matteo Fischetti, and Martin Grötschel, Solving the asymmetric travelling salesman problem with time windows by branch-and-cut, August 1999. Preprint SC 99-31; Norbert Ascheuer, Michael Jünger, and Gerhard Reinelt, A branch & cut algorithm for the asymmetric hamiltonian path problem with precedence constraints. Available via www at www.zib.de, Dec. 1997; Edward K. Baker, An exact algorithm for the time-constrained travelling salesman problem, Operations Research, vol. 31, pp. 938-945, September-October 1983; Rainer E. Burkard, Vladimir G. Deineko, René van Dal, Jack A. A. van˜der Veen, and Gerhard J. Woeginger, Well-solvable special cases of the TSP: A survey, Tech. Rep. 52, Karl-Franzens-Universität & Technische Universität Graz, December 1995; Egon Balas and Matteo Fischetti, A lifting procedure for the asymmetric traveling salesman polytope and a large new class of facets, Mathematical Programming, vol. 58, no. 3, pp. 325-352, 1993; Egon Balas, Matteo Fischetti, and William R. Pulleyblank, The precedence-constrained asymmetric traveling salesman polytope, Mathematical Programming, vol. 68, no. 3, pp. 241-265, 1995; Giovanni Cesari, Divide and conquer strategies for parallel TSP heuristics, Computers & Operations Research, vol. 23, no. 7, pp. 681-694, 1996; Harlan Crowder and Manfred W. Padberg, Solving large-scale symmetric travelling salesman problems to optimality, Management Science, vol. 26, pp. 495-509, March 198, all incorporated by reference herein for all purposes). These methods, solutions, and algorithm are useful for at least some embodiment of the invention to minimize the edges.

In another aspect of the invention, probes very often come in pairs or quadruplets of related probes. These related probes almost always have only one or two edges between them. Thus, it is useful to assign the related probe sets as blocks, rather than individual probes in some embodiments. As used herein, the term block may contain a single probe or related probes or probe sets.

One of skill in the art would appreciate that this is one of many possible weighting functions. Other weighing functions are also within the scope of the invention. For computational efficiency, in one embodiment, only nearby cells need to be counted, since weights for extremely distant cells are negligible.

The edge minimization problem may be solved using a computer to arrange the blocks of probes so that the edge count or weighted edge count is minimal. Normally, there are many features on the chip that may not be moved (control probes, text, spatial normalization features), and these may form constraints on the process of minimization.

One method of solving the edge minimization problem is to use an annealing approach. In this approach, pairs of blocks of probes are swapped at random—if the random swap results in an improvement, it is always kept. If the swap increases the edge count, then the resulting arrangement is kept with a probability dependent upon a hidden variable of Temperature (the temperature is a parameter which controls the bias in optimization towards locally good solutions), otherwise the swap is undone.

Lower (cooler) temperatures reject swaps that increase the edge count more often than higher temperatures. Simulated annealing with properly cooled temperatures is an often-used tool for large optimization problems. However, annealing of arrays takes a long time in practice.

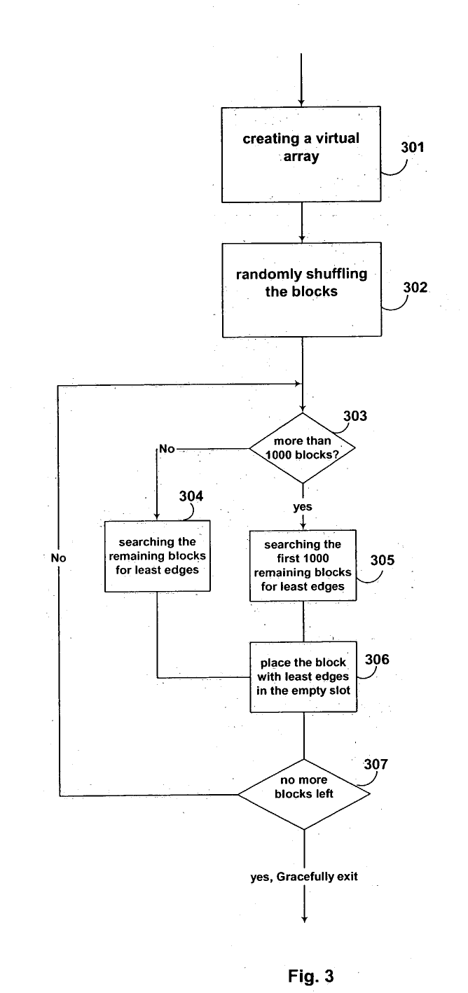

In yet another aspect of the invention, a simpler and faster algorithm employing a locally greedy approach is provided (FIG. 3). A locally greedy approach considers one “slot” on an array, which is a substrate containing spatially arranged polymers such as oligonucleotide probes at a time where a block of probes can be placed. A set of blocks that have not yet been optimized are tried and the optimal (normally the block with the minimal edge count) block is chosen and placed into that slot (displacing the block currently in that slot, if the slot is not empty). This process continues, considering all the slots on the array that have not yet been optimized until all slots have had a “locally best” block placed in them.

In one implementation, all blocks that are valid (i.e. are specified as allowed to be moved by the user) are removed from the array, leaving a set of empty slots to be filled. These slots are then searched in a diagonal fashion, with a user-specified number of blocks specified to search for each slot. Thus, in a two dimensional array, each block typically is compared to previously placed blocks to the “north” and “west” directions, with the “east” and “south” directions consisting of empty slots. One of skill in the art would appreciate that other direction of comparison may also be used.

For example, in one embodiment of computer implemented method, 135,000 blocks consisting of pairs of probes could be found on an expression chip. The order of the blocks is shuffled randomly (FIG. 3, 302), and then the first subset of 1000 blocks (in the computer software product for performing the method, the number of blocks in the subset may be specified by a user, preferably, the number may be in the range of 20-100, 100-500, 500-1000, 1000-10000) are checked against the first slot on the chip (305). The best fitting block (least edge count) is placed into that slot, leaving 134,499 blocks remaining (306). This process continues, moving across the chip adding to empty slots. Towards the end of the chip, when there are fewer than 1000 blocks remaining, only the actual number of blocks remaining are searched when attempting to fill an empty slot (304).

The user specified subset of blocks speeds up the computation by limiting the search to only a few blocks per slot, rather than comparing all the remaining blocks to the current empty slot. There is a cost in the amount of optimization done, but this parameter allows the user to trade off the amount of computation done against the quality of optimization (exact trade-offs depend on the structure of the array). It is of course obvious that the order in which the empty slots are traversed is not crucial, however, experimentation has determined that diagonal replacement works well, with a possible slight advantage over horizontal or vertical replacement.

Computer software products for implementing the locally greedy optimization may contain computer codes for performing each of the steps of the computer implemented methods described above.

In an additional aspect of the invention, methods, systems and computer software products are provided for solving Robust Arrangement Problem (RAP).

Oligonucleotide arrays for monitoring gene expression (See, e.g., U.S. Pat. No. 6,040,138, which is incorporated herein by reference for all for detailed description of using oligonucleotide array for gene expression monitoring) may have certain number of probe pairs (generally a probe that is designed to be complementary to a target gene and a probe that is designed to contain at least one mismatch), such as 10, 15, or 20 probe pairs devoted to any given gene. Local problems (flecks of dust, bubbles, defects) may occur on the array, and if the probe pairs are arranged adjacent to each other, there may be no informative probes remaining for that gene if a defect occurs. The RAP is a probe distribution problem of arranging all the probe pairs on the chip, so that of the N (typically, 10, 15 or 20 pairs) probe pairs associated with any given gene, no more than K, such as 2, 3, 4 or 5, of them are within a radius R of each other. While methods and computer software for solving the RAP problem is described using probe pairs as examples, the methods and computer software is also useful for other probe arrangement. For example, mismatch probes may be unnecessary for gene expression monitoring purpose in some embodiments. In such embodiments, the RAP problem is to reduce non-robust probes rather than adjacent probe pairs.

Typically, for an edge optimized chip using the above-described methods, software or system, the probes are scrambled across the chip, and the probe pairs for a given gene are unlikely to be near each other. However, there may be some positions where K probe pairs for a given gene are within the specified radius R. As used herein, a non-robust (or bad or adjacent) probe pair is a probe pair which occurs as one of the at least K probe pairs associated with a given gene within the specified radius.

In the typical expression array, of the large number of probe-pairs on a chip (>100,000), after edge-optimization, typically fewer than 1% will be non-robust. If all non-robust probe pairs are removed from the chip as blocks, leaving empty slots behind, and an equal number of robust probe pairs are chosen randomly and also removed, and then these blocks are replaced (almost) randomly into the slots, the number of new non-robust blocks will be reduced greatly (typically again cut to 1% of the former value). This dilution procedure may be repeated until there are no non-robust blocks remaining.

Computer software products for solving RAP is also provided (part of edgeopt.cpp, Appendix B). In preferred embodiments, software products may contain both code for performing edge minimization and for solving RAP.

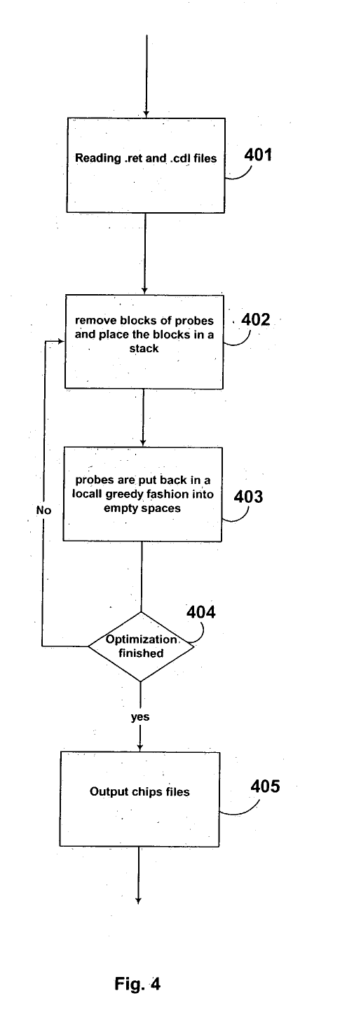

In one embodiment, the basic structure of the computer software for performing the optimization is described as follows (see, also, FIG. 4): .ret and cdl files are read in to describe a chip. Selected blocks of probes (atoms) are removed from the chip and placed on a stack. Empty spaces are left behind. Probes are then put back in a locally greedy fashion into the empty spaces. These steps may be repeated for many different types of blocks. The scrambled chips may then be output to a variety of files.

Appendix A is a computer program in c++ (travel.cpp) that is used to reducing or minimizing the edges between cells using travelling salesman optimization of an ordered list of polymers. The algorithm provides a general insertion heuristic.

Appendix B is a computer program in c++ (edgeopt.cpp) that operate in a locally greedy fashion to optimize the sequence chips in two dimensions. Optimizing chips in two dimensions simultaneously allows for fewer edges on all sides of the probes (more optimization is possible) and for the optimization to be more uniform on all edges of the probes.

Valid commands for Edge Optimization using this exemplary software embodiment are:

lu=lower unit number of range

uu=upper unit number of range

v=value of validflag (1=valid for stripping, 0=don't move)

d=destype

h=height of block/atom (i.e. 2, 4, . . . )

sl=searchlimit=max number of possibilities to search through

r=radius

mn=max allowed

1. Must be first two commands given:

READCDL: in.cdl=read in cdl file

READRET: in.ret=read in ret file

2. Set valid entities for moving:

SETVALIDUNITS: lu uu v

SETVALIDAREA: x y tx ty v

SETVALIDANTIAREA: x y tx ty v

SETVALIDDESTYPE: d

3. Actually put movable blocks onto the stack:

STRIPBLOCKS: h

4. Replace blocks into the allowed space:

DIAGONALREPLACEMENT: sl

HORIZONTALREPLACEMENT: sl

AGGREPLACEMENT: sl

5. Do proximity checking, and fix bad (adjacent) entities:

SETPROXIMITY: r m

FIXBAD: sl

Steps 2-5 may be repeated as needed to optimize different sets of blocks on the chip.

6. Output the data:

DUMPCDL: out.cdl

DUMPRET: out.ret

DUMPMUT: out.mut

LDUMPDIFF: out.dff

7. Exit gracefully:

END:

While the edge minimization methods and software products are described for use in the synthesis of oligonucleotide arrays using VLSIP™ technology employing masks, the method and software products of the invention are also useful for many other purposes including maskless synthesis. For example, the methods and software are useful for VLSIP™ technology employing micro-mirrors instead of masks (U.S. patent application Ser. No. 09/318,775, see also, Signh-Gasson et al., Maskless fabrication of light-directed oligonucleotide microarrays using a digital micromirror array, Nature-Biotechnology 17:974-978, 1999, both incorporated herein by reference for all purposes). It would also be apparent to those with skill in the art that the methods and software products of the invention is also useful for the synthesis of sequence arrays using ink-jet printing or mechanic flow control. More generally, the methods and software products of the invention are useful for the minimization of edges between features.

The above description is illustrative and not restrictive. Many variations of the invention will become apparent to those of skill in the art upon review of this disclosure. Merely by way of example, while the invention is illustrated with particular reference to the evaluation of DNA, the methods can be used in the synthesis and data collection from chips with other materials synthesized thereon, such as RNA and peptides (natural and unnatural). The scope of the invention should, therefore, be determined not with reference to the above description, but instead should be determined with reference to the appended claims along with their full scope of equivalents.

We claim:

1. A computer implemented method for arranging polymers for combinatorial synthesis of said polymers on a substrate comprising:
reducing edge count between said polymers comprising
computer-implemented steps for optimization of an ordered list of polymers.

2. The method of claim 1 wherein said steps for optimization comprises steps for travelling salesman optimization of said ordered list of polymers.

3. The method of claim 2 wherein said travelling salesman optimization is performed by means of a locally greedy insertion heuristic.

4. A computer implemented method for arranging polymers for combinatorial synthesis of said polymers on a substrate comprising:
reducing edge count between said polymers comprising:
dividing said polymers into a plurality of blocks, wherein each of said block comprising one or more related polymers, wherein each of said blocks is to be assigned to one slot on said substrate; and
selecting a subset of said blocks from unassigned blocks; and
assigning one block of said blocks in said set to an empty slot, wherein said one block is the best fitting and results in a least edge count among said blocks of said subset.

5. The method of claim 4 further comprising repeating said steps of selecting and assigning until all blocks are assigned.

6. The method of claim 5 wherein said assigning comprises:
computing a plurality of edge counts, each of said edge counts represents the result of assigning one block of said subset to said empty slot;
comparing said edge counts and selecting said best fitting block, wherein said best fitting block has said least edge count.

7. The method of claim 6 wherein said blocks are ordered randomly and said selecting step comprises selecting the first subset among unassigned blocks.

8. The method of claim 7 wherein the last of said subsets has no more than 100 blocks and other said subset has at least 20 blocks and no more than 100 blocks.

9. The method of claim 7 wherein the last of said subset has no more than 1000 blocks and other said subset has at least 100 blocks and no more than 1000 blocks.

10. The method of claim 7 wherein the last of said subsets has no more than 10000 blocks and other said subset has at least 1000 blocks and no more than 10000 blocks.

11. The method of claim 7 wherein said polymers are oligonucleotides.

12. The method of claim 11 wherein said combinatorial synthesis is radiation directed synthesis.

13. The method of claim 12 wherein said radiation directed synthesis comprises steps of controlling irradiation to active synthesis site using a mask.

14. The method of claim 13 wherein said edge count is a weighted edge count taking into account distance to cell leaking radiation.

15. A computer implemented method for arranging nucleic acid probes in a nucleic acid probe array comprising:
providing an arrangement of said nucleic acid probes;
reducing non-robust probes in said arrangement, wherein said non-robust probe is a probe that occurs as one of at least two (K) probes associated with a given gene within a specified area of said array, comprising:
removing non-robust blocks and optionally removing additional blocks, wherein said non-robust blocks comprises at least one non-robust probe and leaving empty slots in said initial arrangement; and
reassigning said blocks to empty slots of said arrangement.

16. The method of claim 15 wherein said K is at least three.

17. The method of claim 16 wherein said K is at least four.

18. The method of claim 17 wherein said K is at least five.

19. The method of claim 15 wherein said removing step comprises removing said additional blocks randomly.

20. The method of claim 19 wherein said reassigning step comprises reassigning said blocks into said empty slots randomly.

21. The method of claim 20 further comprising repeating steps of removing and reassigning.

22. A computer software product for arranging polymers for combinatorial synthesis of said polymers on a substrate comprising:
code for reducing edge count between said polymers comprising
code for optimizating an ordered list of polymers; and
a computer readable medium for storing said code.

23. The computer software product claim 22 wherein said code for optimizing comprises code for travelling salesman optimization of said ordered list of polymers.

24. The computer software product of claim 23 wherein said code for travelling salesman optimization comprises code for a locally greedy insertion heuristic.

25. A computer software product for arranging polymers for combinatorial synthesis of said polymers on a substrate comprising:
code for reducing edge count between said polymers comprising
code for dividing said polymers into a plurality of blocks, wherein each of said blocks comprises one or more related polymers, and wherein each of said blocks is to be assigned to one slot on said substrate; and
code for selecting a subset of said blocks from unassigned blocks; and
code for assigning one block of said blocks in said set to an empty slot, wherein said one block-is the best fitting and results in a least edge count among said blocks of said subset; and
a computer readable medium for storing said code.

26. The computer software product of claim 25 further comprising code for repeating execution of said codes of selecting and assigning until all blocks are assigned.

27. The computer software product of claim 26 wherein said code for assigning comprises:
code for computing a plurality of edge counts, each of said edge counts represents the result of assigning one block of said subset to said empty slot; and
code for comparing said edge counts and selecting said best fitting block, wherein said best fitting block has said least edge count.

28. The computer software product of claim 27 wherein said blocks are ordered randomly and said code for selecting comprises code for selecting the first subset among unassigned blocks.

29. The computer software product of claim 28 wherein the last of said subsets has no more than 100 blocks and other said subset has at least 20 blocks and no more than 100 blocks.

30. The computer software product of claim 28 wherein the last of said subset has no more than 1000 blocks and other said subset has at least 100 blocks and no more than 1000 blocks.

31. The computer software product of claim 28 wherein the last of said subsets has no more than 10000 blocks and other said subset has at least 1000 blocks and no more than 10000 blocks.

32. The computer software product of claim 28 further comprising code for inputting size of subsets.

33. The computer software product of claim 28 wherein said edge count is a weighted edge count taking into account distance to cell leaking radiation.

34. A computer software product for arranging nucleic acid probes in a nucleic acid probe array comprising:
code for reducing non-robust probes in an arrangement of said probes, wherein said non-robust probe is a probe that occurs as one of at least two (K) probes associated with a given gene within a specified area of said array, comprising:
code for removing non-robust blocks and optionally additional blocks, wherein non-robust blocks comprises at least one robust probe from said arrangement and leaving empty slots in said initial arrangement;
code for reassigning said blocks to empty slots of said arrangement; and
a computer readable medium for storing said codes.

35. The computer software product of claim 34 wherein K is at least three.

36. The computer software product of claim 34 wherein said K is at least four.

37. The computer software product of claim 34 wherein said K is at least five.

38. The computer software product of claim 34 wherein said code for removing comprises code for removing said other blocks randomly.

39. The computer software product of claim 38 wherein said code for reassigning comprises code for reassigning said blocks into said empty slots randomly.

40. The computer software product of claim 34 further comprising code for repeating execution of said codes for removing and reassigning.
